(19) United States
(12) Patent Application Publication
(10) Pub. No.: US 2009/0182222 A1
(43) Pub. Date: Jul. 16, 2009

(54) MAGNETIC RESONANCE IMAGING APPATATUS AND IMAGE RECONSTRUCTION METHOD

(76) Inventors: Yoshio MACHIDA, Nasushiobara-shi (JP); Yoshimori Kassai, Nasushiobara-shi (JP)

Correspondence Address: NIXON & VANDERHYE, PC, 901 NORTH GLEBE ROAD, 11TH FLOOR, ARLINGTON, VA, 22203, US

(21) Appl. No.: 12/349,648

(22) Filed: Jan. 7, 2009

(30) Foreign Application Priority Data
Jan. 10, 2008 (JP) 2008-003480
Nov. 26, 2008 (JP) 2008-301349

(51) Int. Cl. A61B 5/055

(52) U.S. Cl. 600/410

(57) ABSTRACT

In a calculator system of a magnetic resonance imaging apparatus, a filter setting unit sets a shape of a filter superimposed on k-space data to match a shape of a data collecting area in a k-space. A filter processing unit performs a filtering process on the k-space data using the filter of which the shape is set.

### CROSS-REFERENCE TO RELATED APPLICATIONS

This application is based upon and claims the benefit of priority from the prior Japanese Patent Application No. 2008-3480, filed on Jan. 10, 2008, and No. 2008-301349, filed on Nov. 26, 2008; the entire contents of which are incorporated herein by reference.

### BACKGROUND OF THE INVENTION

1. Field of the Invention

The present invention relates to a magnetic resonance imaging apparatus and an image reconstruction method for collecting k-space data concerning an interior of a subject using nuclear magnetic resonance phenomenon and reconstructing an image from the collected k-space data. In particular, the present invention relates to a filtering process performed on the k-space data.

2. Description of the Related Art

Conventionally, a magnetic resonance imaging apparatus collects data concerning an interior of a subject using nuclear magnetic resonance phenomenon and reconstructs an image from the collected data. Specifically, the magnetic resonance imaging apparatus irradiates a radio frequency (RF) wave onto the subject placed in a magnetostatic field, thereby exciting hydrogen nuclei within the subject. The magnetic resonance imaging apparatus detects magnetic resonance signals emitted from the subject as a result of excitation and reconstructs an image from data generated based on the magnetic resonance signals.

In the magnetic resonance imaging apparatus, the data generated from the magnetic resonance signals is arrayed in k-space. Coordinate axes of the k-space are respectively in a read-out (RO) direction and a phase-encode (PE) direction. The data arrayed in the k-space is referred to as “k-space data”. An image indicating actual space is obtained by a predetermined reconstruction process including Fourier transform being performed on the k-space data.

Ordinarily, in the reconstruction process of the image, a filtering process is performed on the k-space data before the Fourier transform to suppress artifacts, such as ringing artifacts (refer to, for example, JP-A H6-327649 (KOKAI)). For example, a “circular filter” that performs isotropic filtering, a “direct product-type filter” that performs filtering based on collected coordinates, and the like are methods used to perform the filtering process on two-dimensional k-space data.

Specifically, the circular filter is a method in which filtering is symmetrically (isotropically) performed from a point of origin in the k-space. A data collection area in the magnetic resonance imaging apparatus is often rectangular. When the data collection area is rectangular, filtering is performed in an elliptical shape. In the circular filter, filtering can be performed on an arbitrary k-space trajectory in the magnetic resonance imaging apparatus. Filtering of three-dimensional k-space data is performed in a spherical shape rather than a circular shape.

On the other hand, the direct product-type filter is a most standard method adhering to a flow of processing in a two-dimensional. Fourier transform (2DFT) method. In the direct product-type filter, a one-dimensional filtering process is successively performed in the RO direction and the PE direction. In a three-dimensional. Fourier transform (3DFT) method, the one-dimensional filtering process is also performed in a slicing direction (referred to, hereinafter, as a slice-encode [SE] direction).

The magnetic resonance imaging apparatus uses various data collection methods, such as a method of collecting data with a shortened front half of an echo to shorten echo time (TE), and a method of collecting less data in either the RO direction or the PE direction to reduce imaging time. A half-Fourier method may or may not be applied when the above-described data collection methods are used. However, regardless of whether the half-Fourier method is applied, additional processing is often performed on areas in which data is insufficient. For example, a filter processed into a sloped shape is applied.

Because the above-described conventional circular filter has an isotropic property, ringing artifacts can be suppressed. However, because k-space data collected from an originally rectangular data collecting area cannot be fully used, resolution decreases. In the direct product-type filter, because properties differ in each coordinate axis direction and diagonal line direction, artifacts occur. In particular, when a waveform of a filter function used in the filtering process has a property of accentuating a intermediate frequency range, because the waveform has a property of further accentuating the intermediate frequency range in a diagonal element direction, the artifacts occur frequently. In the various data collection methods described above, when a filter is processed in correspondence to each method, a design of some sort is required to be individually applied.

### SUMMARY OF THE INVENTION

According to one aspect of the present invention, a magnetic resonance imaging apparatus includes a data collecting unit that collects k-space data concerning an interior of a subject using nuclear magnetic resonance phenomenon; a filter setting unit that sets a shape of a filter superimposed on the k-space data filling a data collecting area in a k-space, such as to match a shape of the data collecting area; a filter processing unit that performs a filtering process on the k-space data collected by the data collecting unit using the filter of which the shape is set by the filter setting unit; and a reconstruction processing unit that reconstructs an image from the k-space data on which the filtering process is performed by the filter processing unit.

According to another aspect of the present invention, a magnetic resonance imaging apparatus includes a data collecting unit that collects k-space data concerning an interior of a subject using nuclear magnetic resonance phenomenon; a filter processing unit that performs a filtering process on the k-space data collected by the data collecting unit, using a filter having a shape that is deformed to become closer to rectangular from circular, the rectangular indicating a data collecting area in a k-space; and a reconstruction processing unit that reconstructs an image from the k-space data on which the filtering process is performed by the filter processing unit.

According to still another aspect of the present invention, an image reconstructing method includes acquiring k-space data concerning an interior of a subject collected using nuclear magnetic resonance phenomenon; setting a shape of a filter superimposed on the k-space data filling a data collecting area in a k-space, such as to match the shape of the data collecting area; performing a filtering process on the acquired k-space data using the filter of which the shape is set; and reconstructing an image from the k-space data on which the filtering process is performed.

According to still another aspect of the present invention, an image reconstructing method includes acquiring k-space data concerning an interior of a subject collected using nuclear magnetic resonance phenomenon; performing a filtering process on the acquired k-space data using a filter having a shape that is deformed to become closer to a shape of a data collecting area in a k-space; and reconstructing an image from the k-space data on which the filtering process is performed.

### BRIEF DESCRIPTION OF THE DRAWINGS

FIG. 1 is a block diagram of an overall configuration of an MRI apparatus according to an embodiment of the present invention;

FIG. 2 is a functional block diagram of configurations of a controlling unit and an image reconstructing unit shown in FIG. 1;

FIGS. 3A to 3C are diagrams illustrating a shape of a filter set by a filter setting unit;

FIG. 4 is a flowchart of a process performed by the MRI apparatus according to the embodiment;

FIG. 5 is an explanatory diagram illustrating when an asymmetrically shaped filter is set;

FIG. 6 is an explanatory diagram illustrating when the asymmetrically shaped filter is set;

FIGS. 7A and 7B are explanatory diagrams illustrating when a one-dimensional filter function is changed between a front half and a latter half of data collection;

FIG. 8 is an explanatory diagram illustrating when the one-dimensional filter function is changed between the front half and the latter half of data collection;

FIG. 9 is an explanatory diagram illustrating when the one-dimensional filter function is changed between the front half and the latter half of data collection;

FIGS. 10A to 10F are explanatory diagrams illustrating a conventional data collection method used in an MRI apparatus;

FIGS. 11A to 11C are diagrams illustrating a shape of a conventional circular filter used in the MRI apparatus;

FIGS. 12A and 12B are diagrams illustrating a shape of the conventional circular filter used in the MRI apparatus;

FIGS. 13A to 13C are diagrams illustrating a shape of a conventional direct product-type filter used in the MRI apparatus; and

FIGS. 14A to 14D are explanatory diagrams illustrating a conventional asymmetrical data collection method used in the MRI apparatus.

### DETAILED DESCRIPTION OF THE INVENTION

Exemplary embodiments of the present invention are below described with reference to the attached drawings. Hereinafter, a magnetic resonance imaging apparatus is referred to as an “MRI apparatus”.

Before a description is given of an MRI apparatus according to an embodiment, conventional data collection methods and a conventional filtering process used in an MRI apparatus will be described in detail.

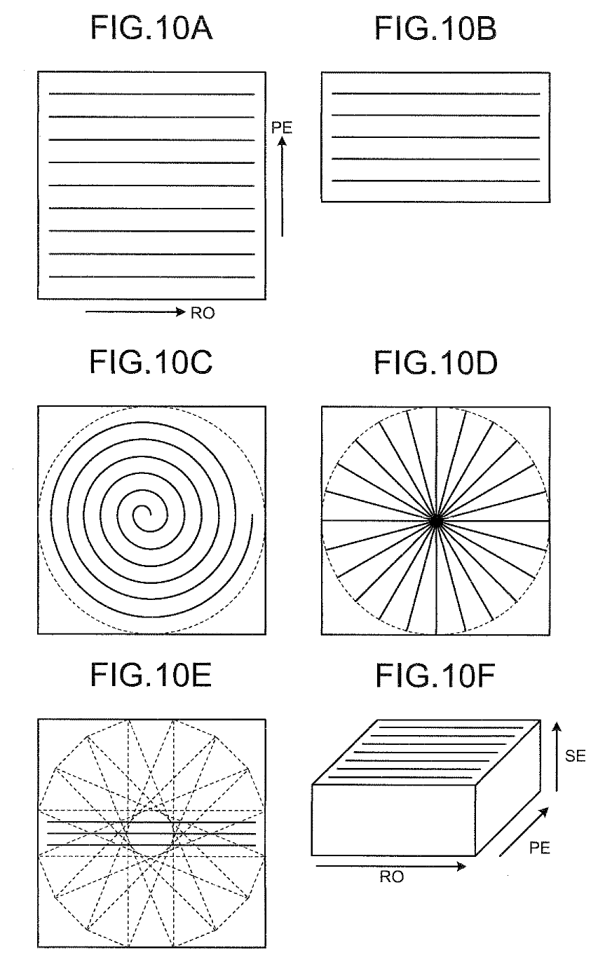

First, conventional data collection methods will be described. FIGS. 10A to 10F are diagrams illustrating the conventional data collection methods used in the MRI apparatus. Conventionally, the MRI apparatus uses various data collection methods. A most standard method is square Cartesian-coordinates collection (two-dimensional Fourier transform method) shown in FIG. 10A and rectangular Cartesian-coordinates collection (two-dimensional Fourier transform method) shown in FIG. 10B, and the like.

In principle, the MRI apparatus can collect data on an arbitrary k-space trajectory by controlling a waveform of a gradient magnetic field. For example, the MRI apparatus can perform spiral collection such as that shown in FIG. 10C, radial collection such as that shown in FIG. 10D, and propeller collection such as that shown in FIG. 10E. Moreover, the MRI apparatus can perform three-dimensional data collection, such as Cartesian-coordinates collection referred to as a three-dimensional Fourier transform method, shown in FIG. 10F.

In the MRI apparatus, spaces shown in FIGS. 10A to 10F are equivalent to a k-space (wave number space) of an image. Therefore, superimposition of a linear filter on the image is widely known to be equivalent to multiplication of a “filter function” in a collected data space. Descriptions are given hereinafter under this assumption.

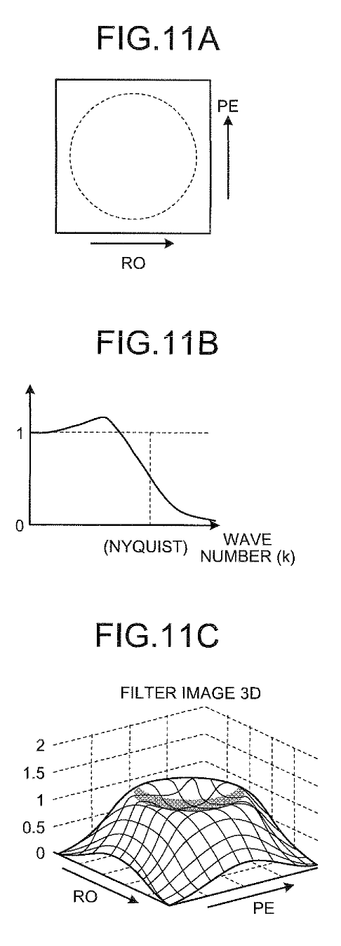

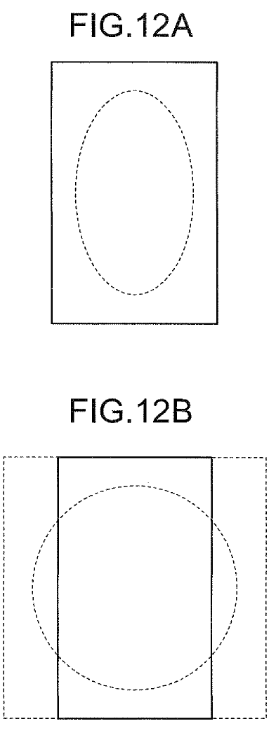

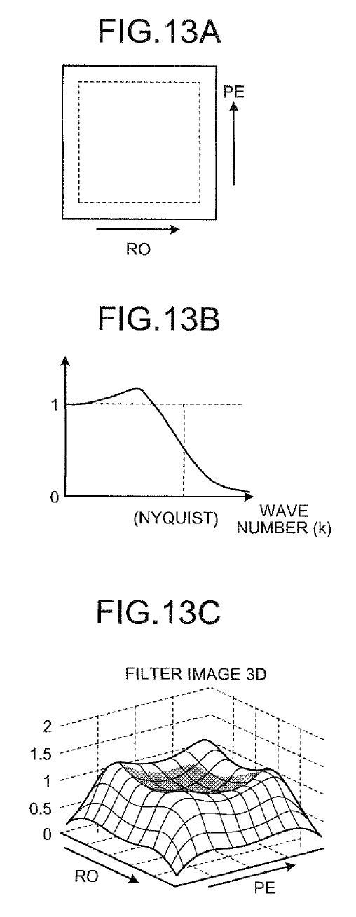

Next, the conventional filtering process will be described. As described earlier, a circular filter and a direct product-type filter are standard filters used in the conventional filtering process. FIGS. 11A to 11C, 12A, and 12B are diagrams illustrating shapes of the conventional circular filter in the MRI apparatus. FIGS. 13A to 13C are diagrams of shapes of the conventional direct product-type filter in the MRI apparatus.

As shown in FIG. 11A, the circular filter is symmetrical in relation to a point of origin of the k-space. The circular filter can be obtained, for example, by a one-dimensional linear filter such as that shown in FIG. 11B being rotated with the point of origin of the k-space as a center, as shown in FIG. 11C. Regardless of which data collection method described above is used, a most natural shape of a filter is the circular filter that is symmetrical in relation to the point of origin, in terms of properties of the k-space.

In particular, the circular filter is extremely natural in radial collection in which data is collected in a radial manner. When the data is collected in a rectangular shape in the k-space, the circular filter may be processed into an elliptical filter as shown in FIG. 12A. Alternatively, the circular shape of the circular filter may be cut-off as shown in FIG. 12B.

On the other hand, as shown in FIG. 13A, the direct product-type filter is applied along coordinate axes of the k-space. The direct product-type filter can be obtained, for example, by a one-dimensional linear filter such as that shown in FIG. 13B being multiplied in a direction of each coordinate axis as shown in FIG. 13C. A two-dimensional Fourier transform process or a three-dimensional Fourier transform process performed as an image reconstruction process of the MRI apparatus is often performed by a one-dimensional Fourier transform process being serially performed for each coordinate axis in implementation. Therefore, it is advantageous for the filtering process to also be performed for each coordinate axis. In this case, the direct product-type filter is used.

In the direct product-type filter, for example, a filter function is a value of 1.2 at a certain point. When a filter of a same shape is applied to two axes, as shown in FIG. 13C, the value is squared and a wave number accentuation of 1.44-fold occurs in the diagonal line direction, thereby disrupting symmetry. In the three-dimensional Fourier transform in particular, 1.2 is cubed such that a wave number accentuation of 1.73-fold occurs, thereby significantly disrupting symmetry.

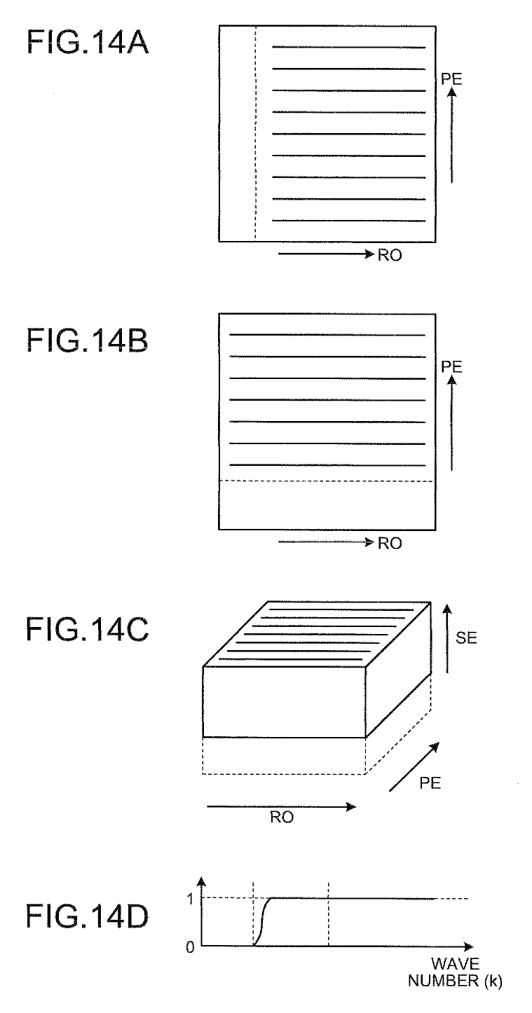

As described earlier, the MRI apparatus may collect data with a shortened front half of an echo to shorten TE. Alternatively, the MRI apparatus may collect less data in one encoding direction to reduce imaging time. In such instances, a data collection area in the k-space becomes asymmetrical. FIGS. 14A to 14D are explanatory diagrams of conventional asymmetrical data collection methods of the MRI apparatus. FIG. 14A indicates when collection from a front half in an RO direction is reduced. FIG. 14B indicates when collection in a PE direction is partially omitted. FIG. 14C indicates when collection in an SE direction is partially omitted.

When a data collection method such as those described above is used, a half-Fourier method may or may not be applied. However, when collected data is used as is, artifacts, such as ringing artifacts, becomes significant. Therefore, conventionally, an area prior to cut-off is processed, such as by multiplication with a function sloped in a trapezoidal shape or a sigmoidal shape, as shown in FIG. 14D.

The data collection methods and the filtering process of the conventional MRI apparatus are as described above. Here, issues regarding the above-described conventional data collection methods and the filtering process are as follows. Because the above-described conventional circular filter has an isotropic property, ringing artifacts can be suppressed. However, because the k-space data collected from an originally rectangular data collecting area cannot be fully used, resolution decreases. An unnecessary portion of data is particularly large when a three-dimensional, spherical filter is applied. In the direct product-type filter, because properties differ in each coordinate axis direction and diagonal line direction, artifacts occur In particular, when a waveform has a property of accentuating a intermediate frequency range, because the waveform has a property of further accentuating the intermediate frequency range in the diagonal line direction, the artifacts occur more easily. In asymmetrical data collection, additional processing is required to be performed on areas in which data is insufficient. For example, a filter processed into a sloped shape is applied.

To solve these issues, in the MRI apparatus according to the embodiment, a shape of the filter is changed depending on the data collecting area in the k-space. As a result, the collected k-space data can be effectively used, and image quality of an image reconstructed from the data can be improved.

Hereafter, the MRI apparatus will be described in detail. In the description below, coordinates in the k-space is expressed as k(kₓ, k_{y}, k_{z}). The k-space data is expressed as F(kₓ, k_{y}, kₓ). Data F basically forms a Fourier transform pair with data f(x, y, z) that is equivalent to an image in an actual space. A filter function is described as H(kₓ, k_{y}, k_{z}).

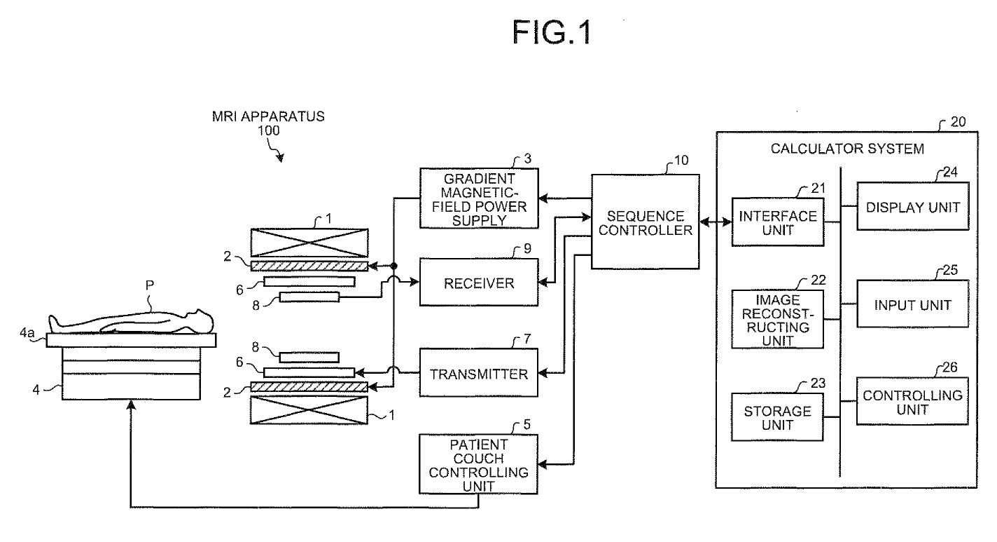

First, an overall configuration of the MRI apparatus according to the embodiment will be described. FIG. 1 is a block diagram of an overall configuration of the MRI apparatus according to the embodiment. An MRI apparatus 100 includes a static magnetic-field magnet 1, a gradient magnetic-field coil 2, a gradient magnetic-field power supply 3, a patient couch 4, a patient couch controlling unit 5, a transmission RF coil 6, a transmitter 7, a reception RF coil 8, a receiver 9, a sequence controller 10, and a calculator system 20.

The static magnetic-field magnet 1 is a hollow, cylindrical magnet that generates a uniform static magnetic field H₀ in a space within the static magnetic-field magnet 1. The static magnetic-field magnet 1 is, for example, a permanent magnet, a superconductive magnet, and the like.

The gradient magnetic-field coil 2 is hollow, cylindrical coil disposed on an inner side of the static magnetic-field magnet 1. The gradient magnetic-field coil 2 is formed by an assembly of three coils corresponding to each axis, X, Y, and Z. The axis X, the axis Y, and the axis Z are perpendicular to one another. The three coils individually receive an electric current supplied by the gradient magnetic-field power supply 3, described hereafter, and generate gradient magnetic fields of which magnetic field intensities change along the axis X, the axis Y, and the axis Z. A Z-axis direction is a same direction as the static magnetic-field magnetic field. The gradient magnetic-field power supply 3 supplies the electric current to the gradient magnetic-field coil 2.

Here, a gradient magnetic field of each axis, X, Y, and Z, generated by the gradient magnetic-field coil 2, respectively correspond, for example, to a slice-selection gradient magnetic-field Gs, a phase-encoding gradient magnetic-field Ge, and a readout gradient magnetic-field Gr. The slice-selection gradient magnetic-field Gs is used to arbitrarily decide an imaging cross-section. The phase-encoding gradient magnetic-field Ge is used to change a phase of a magnetic resonance signal depending on a spatial position. The readout gradient magnetic-field Gr is used to change a frequency of a magnetic resonance signal depending on a spatial position.

The patient couch 4 includes a top plate 4a on which a subject P is placed. Under control of the patient couch controlling unit 5, described hereafter, the top plate 4a is inserted into a cavity (imaging opening) of the gradient magnetic-field coil 2 in a state in which the subject P is placed on the top plate 4a. Ordinarily, the patient couch 4 is set such that a longitudinal direction of the patient couch 4 is parallel with a center axis of the static magnetic-field magnet 1. The patient couch controlling unit 5 controls the patient couch 4. The patient couch controlling unit 5 drives the patient couch 4 and moves the top plate 4a in the longitudinal direction and a vertical direction.

The transmission RF coil 6 is disposed within the gradient magnetic-field coil 2. The transmission RF coil 6 receives a high-frequency pulse from the transmitter 7 and generates a high-frequency magnetic field. The transmitter 7 transmits the high-frequency pulse corresponding to a Larmor frequency to the transmission RF coil 6.

The reception RF coil 8 is disposed within the gradient magnetic-field coil 2. The reception RF coil 8 receives a magnetic resonance signal irradiated from the subject P as a result of an effect of the above-described high-frequency magnetic field. Upon receiving the magnetic resonance signal, the reception RF coil 8 outputs the magnetic resonance signal to the receiver 9.

The receiver 9 generates the k-space data based on the magnetic resonance signal outputted from the reception RF coil 8. Specifically, the receiver 9 generates the k-space data by performing digital conversion on the magnetic resonance signal outputted from the reception RF coil 8. Pieces of information on spatial frequencies in the PE direction, the RO direction, and the SE direction are associated with the k-space data by the above-described slice-selection gradient magnetic-field Gs, the phase-encoding gradient magnetic-field Ge, and the readout gradient magnetic-field Gr. After generating the k-space data, the receiver 9 transmits the k-space data to the sequence controller 10.

The sequence controller 10 drives the gradient magnetic-field power supply 3, the patient couch controlling unit 5, the transmitter 7, and the receiver 9 based on sequence information transmitted from the calculator system 20, thereby performing a scan of the subject P. Here, the sequence information refers to pieces of information defining a procedure for performing the scan, such as an intensity of power supplied by the gradient magnetic-field power supply 3 to the gradient magnetic-field coil 2 and a timing at which the power is supplied, an intensity of an RF signal transmitted from the transmitter 7 to the transmission RF coil 6 and a timing at which the RF signal is transmitted, a timing at which the receiver 9 detects the magnetic resonance signal, and the like.

When, as a result of the sequence controller 10 driving the gradient magnetic-field power supply 3, the patient couch controlling unit 5, the transmitter 7, the receiver 9, and scanning the subject P, the k-space data is transmitted from the receiver 9, the sequence controller 10 transfers the k-space data to the calculator system 20.

The calculator system 20 performs overall control of the MRI apparatus 100, collects data, reconstructs images, and the like. The calculator system 20 includes an interface unit 21, an image reconstructing unit 22, a storage unit 23, a display unit 24, an input unit 25, and a controlling unit 26.

The interface unit 21 controls input and output of various signals exchanged between the calculator system 20 and the sequence controller 10. For example the interface unit 21 transmits the sequence information to the sequence controller 10 and receives the k-space data from the sequence controller 10. Upon receiving k-space data received, the interface unit 21 stores each piece of k-space data in the storage unit 23 for each subject P.

The image reconstructing unit 22 performs post-processing, namely reconstruction such as Fourier transform, on the k-space data stored in the storage unit 23. As a result, the image reconstructing unit 22 generates image data or spectral data of a desired nuclear spin within the subject P. The storage unit 23 stores therein the k-space data received by the interface unit 21, the image data generated by the image reconstructing unit 22, and the like.

The display unit 24 displays various pieces of information, such as the spectral data or the image data under control of the controlling unit 26. A display device such as a liquid crystal display device can be used as the display unit 24. The input unit 25 receives various instructions and pieces of information input by an operator. Pointing devices such as a mouse and a track ball, a selecting device such as a mode selecting switch, and an input device such as a keyboard can be used accordingly as the input unit 25.

The controlling unit 26 including a central processing unit (CPU) (not shown), a memory, and the like performs overall control of the MRI apparatus 100. Specifically, the controlling unit 26 generates the sequence information based on an imaging condition input by the operator through the input unit 25 and transmits the generated sequence information to the sequence controller 10, thereby controlling scanning. The controlling unit 26 also controls the reconstruction of an image based on the k-space data sent from the sequence controller 10 as a result of the scan.

The overall configuration of the MRI apparatus according to the embodiment is described above. Based on a configuration such as this, in the MRI apparatus 100 according to the embodiment, configurations of the controlling unit 26 and the image reconstructing unit 22 of the calculator system 20 are distinct. Specifically, in the calculator system 20, the controlling unit 26 of the calculator system 20 sets the shape of the filter to be superimposed on the k-space data in adherence to a shape of the data collection area in the k-space. When reconstructing an image, the image reconstructing unit 22 performs the filtering process on the k-space data using the filter.

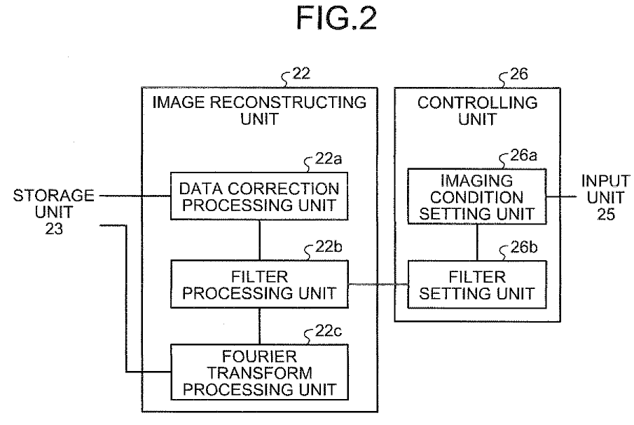

Hereafter, the controlling unit 26 and the image reconstructing unit 22 will be described in detail. FIG. 2 is a functional block diagram of the configurations of the controlling unit 26 and the image reconstructing unit 22 shown in FIG. 1. The controlling unit 26 includes an imaging condition setting unit 26a and a filter setting unit 26b as functional units related to the present invention.

The imaging condition setting unit 26a receives the imaging condition input by the operator through the input unit 25 and generates the sequence information based on the received imaging condition. The sequence information generated by the imaging condition setting unit 26a is transmitted to the sequence controller 10 via the interface unit 21 shown in FIG. 1.

The filter setting unit 26b sets the shape of the filter superimposed on the k-space data based on the imaging condition received by the imaging condition setting unit 26a. Specifically, when the imaging condition input by the operator is received by the imaging condition setting unit 26a, the filter setting unit 26b generates a filter function H adhering to the shape of the data collecting area based on the imaging condition. The filter setting unit 26b sets the filter using the generated filter function H.

For example, when the data collecting area is a two-dimensional space area, the filter setting unit 26b sets the shape of the filter to match the shape of the data collecting area as shown below, by defining a distance from the point of origin in the k-space and expanding a predetermined one-dimensional filter function in association with the distance.

According to the embodiment, a filter function similar to the one-dimensional linear filter shown in FIG. 11B is used as the one-dimensional filter function. The filter function is expressed, hereinafter, as H0(k), where k≧0.

Here, a filter function H0 is ordinarily defined such as to achieve a shape in which damping is performed at a data collection cut-off frequency (ordinarily equivalent to a Nyquist frequency) to control ringing artifacts. When the filter function H0 is defined as a shape such as this, blurring of the image increases as a side effect. Therefore, the filter function H0 is defined such that a intermediate frequency range is slightly accentuated in adherence to image property or a purpose of diagnosis.

According to the embodiment, the distance from the point of origin in the k-space is defined using a vector norm of which a starting point is the point of origin in the k-space. The norm is defined by an expression (1) below.

$${‖ k ‖}_{α} = {( {k}_{x}^{α} + {k}_{y}^{α} )}^{\frac{1}{α}} \qquad ( 1 )$$

In the expression (1), a is set depending on an imaging type, an imaging purpose, and the like, based on the imaging condition. The norm defined by the expression (1) is referred to hereinafter as an “α norm”.

According to the embodiment, the filter function H is defined as a result of the one-dimensional filter function H0 being associated with the α norm, as in an expression (2) below.

H(kₓ, k_{y})=H0(∥k∥_{α}) (2)

For example, the conventional circular filter is equivalent to when α=2 in the above-described expression (2), and is defined by expressions (3) and (4) below.

H(kₓ, k_{y})=H0(∥k∥) (3)

∥k∥=\sqrt{kₓ²+k_{y}²} (4)

The conventional direct product-type filter is defined by an Expression (5) below.

H(kₓ, k_{y})=H0(kₓ)·H0(k_{y}) (5)

Based on definitions such as these, when the imaging condition setting unit 26a receives the imaging condition, the filter setting unit 26b decides a value of α in adherence to the shape of the data collecting area depending on the imaging type and the imaging purpose, based on the imaging condition. Then, the filter setting unit 26b generates the two-dimensional filter function H by assigning the decided value of α to the expression (1) and the expression (2), defined above. The filter setting unit 26b uses the generated filter function H and sets the shape of the filter superimposed on the k-space data.

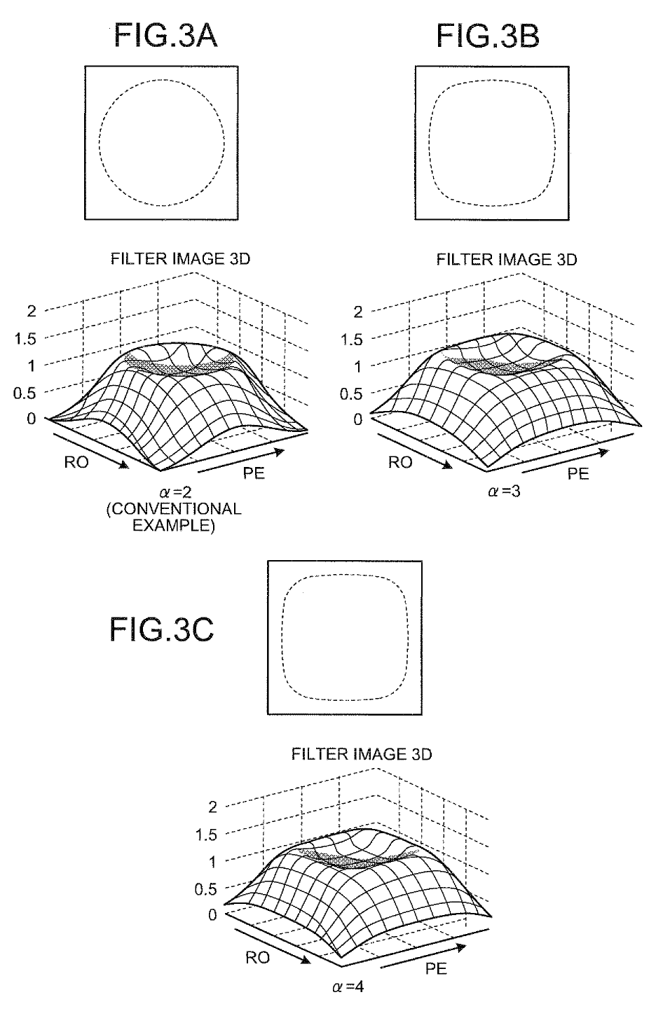

FIGS. 3A to 3C are diagrams illustrating the shapes of the filter set by the filter setting unit 26b. In FIGS. 3A to 3C, each top diagram shows a curved line (a circle slightly deformed to form a rectangle) of which the α norm from the point of origin is a constant value. Each bottom diagram shows a overhead view of the filter function H.

As shown in FIG. 3A, when α=2, the shape of the filter is a circle similar to that of the conventional circular filter. Then, as shown in FIGS. 3B and 3C, when the value of α is 3 or 4, the circular shape of the filter becomes more rectangular as the value of α increases.

In this way, as a result of the filter setting unit 26b setting the shape of the filter to become closer to rectangular from circular, the k-space data can be effectively used in the diagonal line direction as well. Unlike in the conventional direct product-type filter, the intermediate frequency range in the diagonal line direction is not accentuated. As a result of the value of α being changed, the shape of the filter can easily match that of the data collecting area.

Here, the filter setting unit 26b sets the shape of the filter based on the imaging condition set when imaging is performed. Ordinarily, in imaging performed by the MRI apparatus, various imaging conditions are set when the imaging is performed. Various pieces of information are set as the imaging conditions, such as an imaging region, a pulse sequence type used in the imaging, a field of view (FOV), an imaging cross-section type, a number of imaging cross-sections, and a thickness of the imaging cross-section. The filter setting unit 26b sets the shape of the filter based on these pieces of information.

When the shape of the filter is set based on the imaging region, for example, when the imaging region is a head portion, the filter setting unit 26b sets α to 2 such that the shape of the filter is circular. When the imaging region is an abdominal area, the filter setting unit 26b sets α to 4 such that the shape of the filter is more rectangular than that when the imaging region is the head portion.

When the shape of the filter is set based on the imaging cross-section type, for example, when the imaging cross-section is an axial cross-section, the filter setting unit 26b sets α to 2 such that the shape of the filter is circular. When the image cross-section is a sagittal cross-section or coronal cross-section, the filter setting unit 26b sets α to 3 or 4 such that the shape of the filter is more rectangular than that when the cross-section is the axial cross-section. Which value of 3 or 4 set to α is decided by using a filling rate of an imaging object to a FOV. For example, when the filling rate is high for the FOV, α is set to 4.

Here, when the data collecting area is a two-dimensional space area is described. However, even when the data collecting area is a three-dimensional space area, a three-dimensional filter can. be similarly set as a result of a three-dimensional filter function being defined by expression (6) and expression (7) below.

$$\begin{matrix}H ( {k}_{x} , {k}_{y} , {k}_{z} ) = H 0 ( {‖ k ‖}_{α} ) & ( 6 ) \\ {‖ k ‖}_{α} = {( {k}_{x}^{α} + {k}_{y}^{α} + {k}_{z}^{α} )}^{\frac{1}{α}} & ( 7 )\end{matrix}$$

Returning to FIG. 2, the image reconstructing section 22 has a data correction processing unit 22a, a filter processing unit 22b, and a Fourier transform processing unit 22c as functional units related to the present invention. The data correction processing unit 22a performs a predetermined correction process on the k-space data stored in the storage section 23 when the image is reconstructed.

The filter processing unit 22b performs the filtering process on the k-space data on which the correction process has been performed by the data correction processing unit 22a. Specifically, the filter processing unit 22b performs the filtering process on the k-space data when the image is reconstructed, using the filter of which the shape has set by the filter setting unit 26b.

In this way, as a result of the filter processing unit 22b performing the filtering process using the filter formed by the filter setting unit 26b such as to match the shape of the data collecting area, the image can be reconstructed through effective use of the collected k-space data.

The Fourier transform processing unit 22c performs the two-dimensional Fourier transform process or the three-dimensional Fourier transform process on the k-space data filtered by a filter processing unit 2b. As a result, the Fourier transform processing unit 22c generates the image data or the spectral data of the predetermined nuclear spin within the subject P.

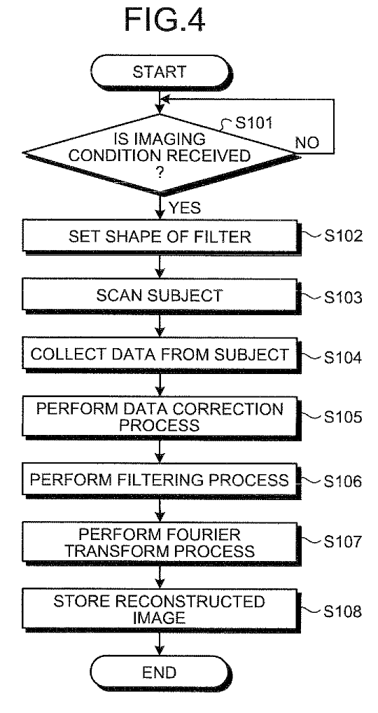

Next, processes performed by the MRI apparatus 100 according to the embodiment will be described. FIG. 4 is a flowchart of the processes performed by the MRI apparatus 100 according to the embodiment. Here, processes performed by the calculator system 20 will mainly be described.

As shown in FIG. 4, in the calculator system 20 of the MRI apparatus 100 according to the embodiment, when the imaging condition setting unit 26a receives the imaging condition from the operator through the input unit 25 (Yes at Step S101), the filter setting unit 26b sets the shape of the filter superimposed on the k-space data based on the imaging condition received by the imaging condition setting unit 26a (Step S102).

Subsequently, based on the sequence information generated by the imaging condition setting unit 26a, the gradient magnetic-field power supply 3, the transmitter 7, and the receiver 9 are driven in adherence to instructions from the sequence controller 10, and the subject P is scanned (Step S103). Then, the interface unit 21 receives the magnetic resonance signal obtained by the receiver 9 via the sequence controller 10 and stores received magnetic resonance signal in the storage unit 23 as the k-space data (Step S104).

Next, the data correction processing unit 22a performs the data correction processing on the k-space data (Step S105). The filter processing unit 22b performs the filtering process on the k-space data (Step S106).

The Fourier transform processing unit 22c then performs the two-dimensional Fourier transform or the three-dimensional Fourier transform on the k-space data on which the filtering process has been performed, thereby reconstructing a two-dimensional image or a three-dimensional image (Step S107). Then, the Fourier transform processing unit 22c stores the reconstructed image in the storage unit 23 (Step S108).

As described above, according to the embodiments the filter setting unit 26b sets the shape of the filter superimposed on the k-space data to match the shape of the data collection area in the k-space. When the image is reconstructed, the filter processing unit 22b performs filtering process on the k-space data using the filter of which the shape has been set. Therefore, the collected k-space data can be effectively used, and the image quality of the reconstructed image can be improved.

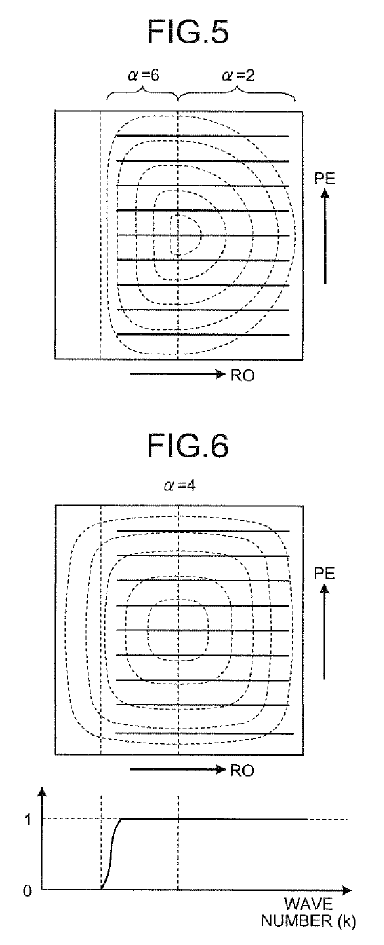

According to the embodiment, when the shape of the data collecting area in the K-space is a shape symmetrical in relation to the coordinate axes is described. However, as described earlier, the data collecting area in the k-space may be an asymmetrical shape depending on the type of data collection method. The present invention can be similarly applied to such instances as well. FIGS. 5 and 6 are diagrams explaining when a filter with an asymmetrical shape is set.

In this instance, for example, the filter setting unit 26b sets the filter having a shape asymmetrical in relation to the coordinate axes of the k-space. Specifically, for example, when few pieces of data are collected from the front half in the RO direction, as shown in FIG. 5, the filter setting unit 26b normalizes coordinates at a length of a section from which the pieces of data has been collected regarding the front half at which the data tends to be insufficient. The filter setting unit 26b also sets the value of α to a larger value when the α norm is determined. In the example in FIG. 5, the value of α is 6 regarding the front half and 2 regarding a latter half.

Alternatively, as shown in a top diagram in FIG. 6, the filter setting unit 26b determines the α norm including sections from which data has not been collected. Subsequently, the filter setting unit 26b multiplies a conventionally used sloped-shape function shown in a bottom diagram of FIG. 6 to the generated filter function. In the example in FIG. 6, the value of α is 4.

Ringing artifacts that occurs as a result of cut-off of the front half at which the data tends to be insufficient is often noticeable in the reconstructed image. Therefore, the filter is preferably set such that only a simple damping process is performed, without the intermediate frequency range being accentuated. Therefore, for example, the shape of the first-dimension filter function can be changed between the front half and the latter half.

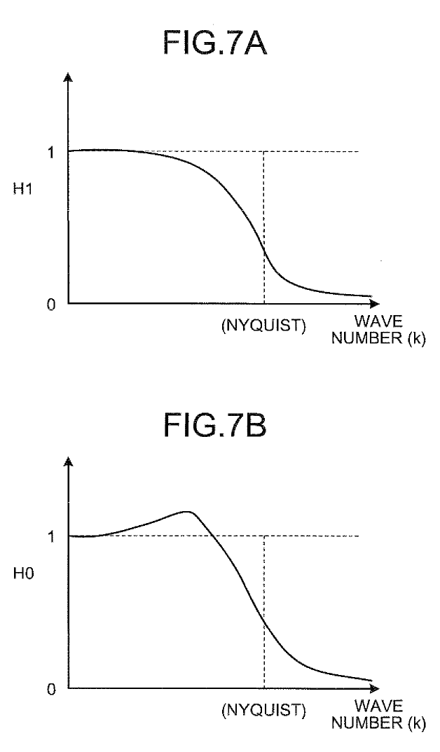

FIGS. 7A, 7B, 8, and 9 are explanatory diagrams of when the one-dimensional filter function is changed between the front half and the latter half of the data collection. In this instance, for the front half in the RO direction in the k-space, the filter setting unit 26b sets the filter using a filter function H1 to which only the damping process is applied, as shown in FIG. 7A. For the latter half, the filter setting unit 26b sets the filter using the filter function H0 to which processing, such as a intermediate frequency range accentuation, is applied, as shown in FIG. 7B.

To set a continuous filter function in the overall k-space using the two filter functions H1 and H0, the filter setting unit 26b temporarily normalizes the coordinates of the k-space to −1≦kₓ≦1 and −1≦k_{y}≦1.

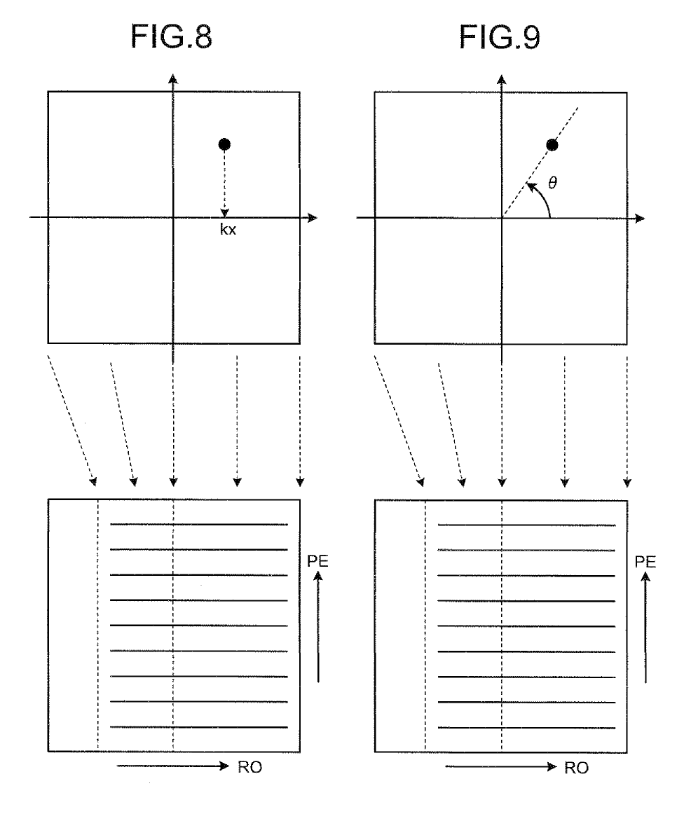

Then, as shown in FIG. 8, the filter setting unit 26b defines the filter function H using an expression (8) below, with u=kₓ/2+(½)

H(kₓ, k_{y})=u×H0(∥k∥_{α})+(1−u)×H1(∥k∥_{α}) (8)

Alternatively, as shown in FIG. 9, the filter setting unit 26b can define the filter function H using the expression (9) below, with θ=arg(k) and u=cos θ/2+(½).

H(kₓ, k_{y})=u×H0(∥k∥_{α})+(1−u)×H1(∥k∥_{α}) (9)

In this way, after determining the filter function in a temporarily normalized space, the filter setting unit 26b moves the filter function to an asymmetrical space and uses the filter function. In other words, in the example, the filter setting unit 26b recreates H(kₓ, k_{y}) by rescaling coordinates in a space kₓ<0 (space in the front half).

As described above, the k-space can be more effectively used even when the shape of the data collection area is asymmetrical in the RO direction, because the filter setting unit 26b sets the shape of the filter from circular to a more rectangular shape asymmetrical in relation to the coordinate axes of the k-space.

When the data in the front half in the RO direction is slightly insufficient is described above. However, the present invention is not limited thereto. Data processing can be similarly performed when the data is insufficient in the PE direction., and even when the data collecting area is three-dimensional.

When the three-dimensional filter is generated, two dimensions are generated at the above-described steps. A remaining one dimension (ordinarily the SE direction) can be the direct product-type filter. In this case, the shape of the one-dimensional filter is preferably that in which the intermediate frequency range is slightly accentuated in a two-dimensional direction., as shown in FIG. 7B. Moreover, the shape of the one-dimensional filter is preferably that in which the intermediate frequency range is not accelerated in the SE direction, as shown in FIG. 7A.

According to the embodiment, when the data collecting area in the k-space is expressed by Cartesian coordinates is mainly described. For example, in the data collection method referred to as the propeller method (refer to FIG. 10E), each blade is a rotated rectangular Cartesian-coordinates collection. Therefore, the above-described method can be applied to each blade, as a result of the filter function being applied in which the distance is defined by the α norm.

In this way, in the MRI apparatus according to the embodiment, the collected k-space data can be effectively used and the image quality of the reconstructed image can be improved in various data collection methods, as a result of the one-dimensional filter being expanded to multiple dimensions using the α norm.

For example, in the spiral collection shown in FIG. 10C, the radial collection shown in FIG. 10D, the propeller collection shown in FIG. 10E, and the like, the collected data fills the k-space such as to be symmetrical in relation to the center of the k-space. Therefore, in these methods, the shape of the filter is preferably made circular by α=2. On the other hand, in the Cartesian-coordinates collection shown in FIGS. 10A and 10B, the collected data is collected such as to be symmetrical in relation to the coordinate axis of the Cartesian coordinates. Therefore, for example, as a result of, the shape of the filter is preferably made more rectangular by α=3 or α=4. In this way, for example, the filter setting unit 26b can set the shape of the filter depending on a method by which the data fills the k-space, the method being set as the imaging condition.

As described above, in the MRI apparatus according to the embodiment, the collected k-space data can be effectively used even when the data collection method varies. Therefore, the filter shape can be flexibly generated. Artifacts can be suppressed, and blurring can be reduced. Moreover, through improvement of image quality, diagnostic capability can be improved.

Each constituent element of each device shown in the diagrams according to the embodiment is a functional concept, and does not necessarily have to be physically configured as shown in the diagrams. In other words, specific aspects of distribution and integration of each device are not limited to those shown in the diagrams. All or some constituent elements can be functionally or physically distributed and integrated in arbitrary units depending on various load and usage conditions.

As described above, the magnetic resonance imaging apparatus and the image reconstruction method of the invention are advantageous for when the k-space data is filtered during reconstruction of the image. In particular, the magnetic resonance imaging apparatus and the image reconstruction method are suitable for when the collected k-space data is required to be effectively used and the image quality of the reconstructed image is required to be improved.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A magnetic resonance imaging apparatus comprising:
a data collecting unit that collects k-space data concerning an interior of a subject using nuclear magnetic resonance phenomenon;
a filter setting unit that sets a shape of a filter superimposed on the k-space data filling a data collecting area in a k-space, such as to match a shape of the data collecting area;
a filter processing unit that performs a filtering process on the k-space data collected by the data collecting unit using the filter of which the shape is set by the filter setting unit; and
a reconstruction processing unit that reconstructs an image from the k-space data on which the filtering process is performed by the filter processing unit.

2. The apparatus according to claim 1, wherein the filter setting unit sets a shape of the filter to become closer to rectangular from circular.

3. The apparatus according to claim 1, wherein the filter setting unit sets a shape of the filter to become closer to asymmetrical rectangular with respect to an axis of the k-space from circular.

4. The apparatus according to claim 1, wherein the filter setting unit generates a two-dimensional filter function to match the shape of the data collecting area, and sets a shape of the filter using the generated filter function.

5. The apparatus according to claim 2, wherein the filter setting unit generates a two-dimensional filter function to match the shape of the data collecting area, and sets a shape of the filter using the generated filter function.

6. The apparatus according to claim 1, wherein the filter setting unit generates a three-dimensional filter function to match the shape of the data collecting area, and sets a shape of the filter using the generated filter function.

7. The apparatus according to claim 2, wherein the filter setting unit generates a three-dimensional filter function to match the shape of the data collecting area, and sets a shape of the filter using the generated filter function.

8. The apparatus according to claim 1, wherein the filter setting unit generates a filter function as a direct product between a two-dimensional filter function and a one-dimensional filter function to match the shape of the data collecting area, and sets a shape of the filter using the generated filter function.

9. The apparatus according to claim 2, wherein the filter setting unit generates a filter function as a direct product between a two-dimensional filter function and a one-dimensional filter function to match the shape of the data collecting area, and sets a shape of the filter using the generated filter function.

10. The apparatus according to claim 1, wherein the filter setting unit defines a distance from a point of origin in the k-space, and sets a shape of the filter by expanding a one-dimensional filter function in association with the defined distance.

11. The apparatus according to claim 2, wherein the filter setting unit defines a distance from a point of origin in the k-space, and sets a shape of the filter by expanding a one-dimensional filter function in association with the defined distance.

12. The apparatus according to claim 10, wherein the fitter setting unit expands the one dimensional filter function to multiple dimensions in association with the distance.

13. The apparatus according to claim 10, wherein the filter setting unit defines α norm of a vector of which a starting point is the point of origin of the k-space, and defines the distance by the norm.

14. The apparatus according to claim 1, wherein the filter setting unit sets the shape of the filter based on an imaging condition set when an imaging is performed.

15. The apparatus according to claim 14, wherein the filter setting unit sets the shape of the filter in accordance with an imaging region set as the imaging condition.

16. The apparatus according to claim 14, wherein the filter setting unit sets the shape of the filter in accordance with a kind of an imaging cross-section set as the imaging condition.

17. The apparatus according to claim 14, wherein the filter setting unit sets the shape of the filter in accordance with a method by which the data fills the k-space set as the imaging condition.

18. A magnetic resonance imaging apparatus comprising:
a data collecting unit that collects k-space data concerning an interior of a subject using nuclear magnetic resonance phenomenon;
a filter processing unit that performs a filtering process on the k-space data collected by the data collecting unit, using a filter having a shape that is deformed to become closer to rectangular from circular, the rectangular indicating a data collecting area in a k-space; and
a reconstruction processing unit that reconstructs an image from the k-space data on which the filtering process is performed by the filter processing unit.

19 An image reconstructing method comprising:
acquiring k-space data concerning an interior of a subject collected using nuclear magnetic resonance phenomenon;
setting a shape of a filter superimposed on the k-space data filling a data collecting area in a k-space, such as to match the shape of the data collecting area;
performing a filtering process on the acquired k-space data using the filter of which the shape is set; and
reconstructing an image from the k-space data on which the filtering process is performed.

20. An image reconstructing method comprising:
acquiring k-space data concerning an interior of a subject collected using nuclear magnetic resonance phenomenon;
performing a filtering process on the acquired k-space data using a filter having a shape that is deformed to become closer to a shape of a data collecting area in a k-space; and
reconstructing an image from the k-space data on which the filtering process is performed.
